(19) United States
(12) Patent Application Publication
(10) Pub. No.: US 2026/0256437 A1
(43) Pub. Date: Sep. 3, 2026

(54) RADIOGRAPHY APPARATUS, METHOD OF OPERATING RADIOGRAPHY APPARATUS, AND PROGRAM OF OPERATING RADIOGRAPHY APPARATUS

(71) Applicant: FUJIFILM CORPORATION, Tokyo (JP)

(72) Inventors: Hisatsugu HORIUCHI, Kanagawa (JP); Tatsuya TANEICHI, Kanagawa (JP); Takeyasu KOBAYASHI, Kanagawa (JP); Naoyuki NISHINO, Kanagawa (JP)

(21) Appl. No.: 19/548,961

(22) Filed: Feb. 25, 2026

(30) Foreign Application Priority Data
Feb. 28, 2025 (JP) 2025-032194

(51) Int. Cl. A61B 6/00

(52) U.S. Cl. CPC A61B 6/4405; A61B 6/547

(57) ABSTRACT

A radiography apparatus is used for radiography, and the radiography apparatus includes: a radiation source or a radiographic image detection device; a driving mechanism that is autonomously drivable and that includes an environment information sensor which acquires surrounding environment information; and a processor that controls the driving mechanism and that is capable of controlling an information acquisition frequency of the environment information sensor according to a driving state of the driving mechanism.

### CROSS-REFERENCE TO RELATED APPLICATIONS

The present application claims priority under 35 U.S.C. §119 to Japanese Patent Application No. 2025-032194, filed on Feb. 28, 2025. The above application is hereby expressly incorporated by reference, in its entirety, into the present application.

### BACKGROUND

### 1. Technical Field

The technology of the present disclosure relates to a radiography apparatus, a method of operating the radiography apparatus, and a program of operating the radiography apparatus.

### 2. Description of the Related Art

In the medical field, a radiography apparatus including a driving mechanism is known. As such a radiography apparatus, for example, a radiation generation apparatus is known in which a body part including a radiation source that emits radiation toward a subject is mounted on a carriage unit including wheels. For example, the radiation generation apparatus is used for so-called ward round imaging in which a patient as a subject is imaged while moving around a ward.

WO2017/043040A discloses a radiation generation apparatus that is manually drivable and includes a camera that images a surrounding environment. In WO2017/043040A, the manual driving to a target position such as a side of a decubitus imaging table (bed) on which an electronic cassette is installed is assisted by notifying an operator such as a radiologic technologist of a path to avoid an obstacle captured by the camera.

### SUMMARY

The inventors have considered adopting a simultaneous localization and mapping (SLAM) method in which autonomous driving to a target position is performed using, for example, a SLAM technology in such a driving type radiography apparatus. In a case in which the autonomous driving can be performed, it is possible to further reduce a burden on the operator.

The SLAM method is a technology of creating a map by recognizing a surrounding environment and estimating a self-position based on the map in parallel, and as an environment information sensor that acquires the surrounding environment information, for example, a camera is used. A processor that executes driving control related to the driving mechanism repeats updating a map of a surrounding environment and estimating a self-position on the map based on an image acquired by the camera. In order to realize accurate driving control to the target position, the accuracy of the self-position estimation is important.

The camera acquires a plurality of images at a frame rate. The frame rate defines an information acquisition frequency of the environment information. The processor executes the driving control by detecting a movement amount of the driving mechanism from a change amount between the plurality of images continuously acquired at the frame rate and estimating the self-position. In the driving control, in a case in which the change amount of the image per unit time is large, in a case in which the frame rate is too low, the change amount between the images is excessively large, and it may take time to search for the self-position, and the estimation accuracy may be decreased. On the other hand, in a case in which the change amount of the image per unit time is small, in a case in which the frame rate is too high, the change amount of the image per unit time is excessively small, and for example, there is a case in which the movement is slightly performed, but the movement is erroneously determined to be stopped.

In such driving control, in a case in which the information acquisition frequency such as the frame rate is always constant, the estimation accuracy of the self-position may be decreased.

The present disclosed technology provides a radiography apparatus, an operation method of a radiography apparatus, and an operation program of a radiography apparatus that can improve the accuracy of the self-position estimation as compared with the related art in a case in which driving control related to an autonomously driving mechanism is executed.

A radiography apparatus according to the present disclosed technology is a radiography apparatus which is used for radiography, the radiography apparatus comprising: a radiation source or a radiographic image detection device; a driving mechanism that is autonomously drivable and that includes an environment information sensor which acquires surrounding environment information; and a processor configured to control the driving mechanism and that is capable of controlling an information acquisition frequency of the environment information sensor according to a driving state of the driving mechanism.

The driving state may include at least one of a movement direction or a moving speed.

The driving state may include straight movement in which the movement direction does not change and rotational movement in which the movement direction changes, and the straight movement may include forward and backward movement, lateral movement, and diagonal movement.

In a case of the rotational movement, the processor may set the information acquisition frequency to be higher than the information acquisition frequency in a case of the straight movement.

In a case of the lateral movement or the diagonal movement, the processor may set the information acquisition frequency to be higher than the information acquisition frequency in a case of the forward and backward movement.

In a case where the rotational movement and the straight movement are performed in parallel, the processor may set the information acquisition frequency to be higher than the information acquisition frequency in a case where only the straight movement is performed.

The processor may control the driving mechanism such that a final position adjustment to a target position is the straight movement.

The processor may increase the information acquisition frequency to be higher as the moving speed is faster.

The processor may acquire a fixed-viewpoint image representing a surrounding environment captured by a fixed-point camera provided at a fixed position, and may execute driving control based on environment information acquired from the environment information sensor and the fixed-viewpoint image.

The environment information sensor may be a camera that images a surrounding environment, and the information acquisition frequency may be a frame rate.

The radiography apparatus may be a radiation generation apparatus including the radiation source.

An operation method of a radiography apparatus according to the present disclosed technology is an operation method of a radiography apparatus which is used for radiography, and which includes a radiation source or a radiographic image detection device, a driving mechanism that is autonomously drivable and that includes an environment information sensor which acquires surrounding environment information, and a processor configured to control the driving mechanism, the operation method comprising: controlling, by the processor, an information acquisition frequency of the environment information sensor according to a driving state of the driving mechanism.

An operation program of a radiography apparatus according to the present disclosed technology is an operation program of a radiography apparatus which is used for radiography, and which includes a radiation source or a radiographic image detection device, a driving mechanism that is autonomously drivable and that includes an environment information sensor which acquires surrounding environment information, and a processor configured to control the driving mechanism, the operation program causing the processor to execute a process comprising: controlling an information acquisition frequency of the environment information sensor according to a driving state of the driving mechanism.

According to the present disclosed technology, the accuracy of the self-position estimation can be improved as compared with the related art in a case in which driving control related to an autonomously driving mechanism is executed.

### BRIEF DESCRIPTION OF THE DRAWINGS

FIG. 1 is a diagram showing a state of decubitus imaging using a radiography system.

FIG. 2 is a diagram showing a state of upright imaging using the radiography system.

FIG. 3 is a diagram showing a radiation generation apparatus in which a radiation source is in an accommodation position.

FIG. 4 is a block diagram showing an electric configuration of the radiation generation apparatus.

FIG. 5 is a diagram showing a state in which the radiation generation apparatus drives from a standby position toward a first target position in the decubitus imaging and stops at the first target position.

FIG. 6 is a diagram showing a state in which the radiation generation apparatus drives from the standby position toward a second target position in the upright imaging and stops at the second target position.

FIG. 7 is a diagram showing processing of each processing unit of a processor related to the driving control.

FIG. 8 is a diagram showing processing of each processing unit of the processor related to the driving control following FIG. 7.

FIG. 9 is a diagram showing processing of each processing unit of a processor related to the alignment control.

FIG. 10 is a diagram showing processing in a learning phase of a cassette contour extraction model.

FIG. 11 is a diagram showing a state of the alignment control.

FIG. 12 is a diagram showing an alignment support screen.

FIG. 13 is a diagram showing contents of the imaging control.

FIG. 14 is a conceptual diagram of controlling a frame rate in the driving control.

FIG. 15 is a diagram showing the straight movement.

FIG. 16 is a diagram showing the rotational movement.

FIG. 17 is a diagram showing the lateral movement.

FIG. 18 is a diagram showing a specific example of the control of the frame rate.

FIG. 19 is a diagram showing the frame rate.

FIG. 20 is a graph showing a relationship between the moving speed and the frame rate.

FIG. 21 is a conceptual diagram of controlling the frame rate according to the moving speed.

FIG. 22 is a diagram showing the final position adjustment.

FIG. 23 is a diagram showing an example of using the fixed-viewpoint image.

### DETAILED DESCRIPTION

### First Embodiment

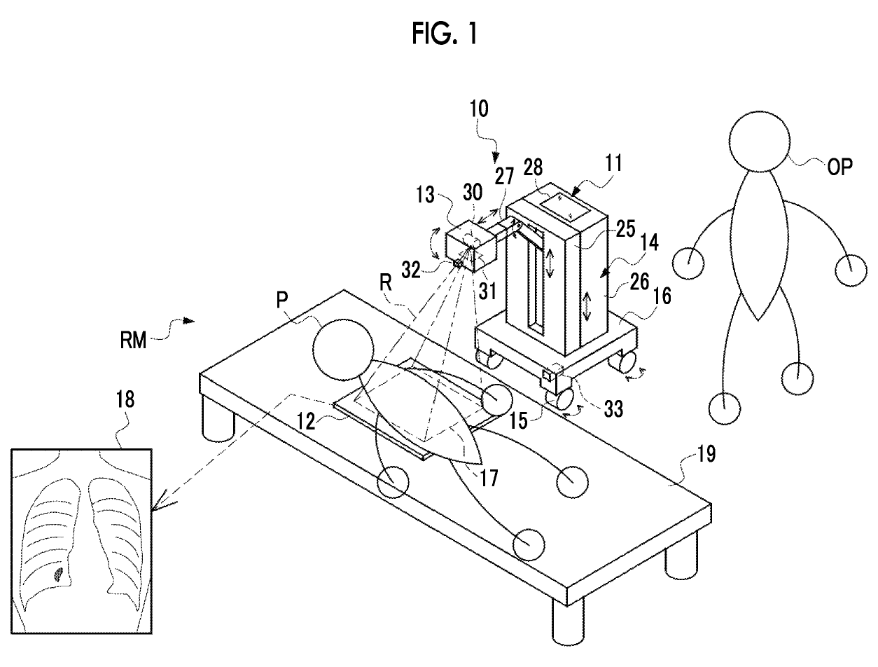

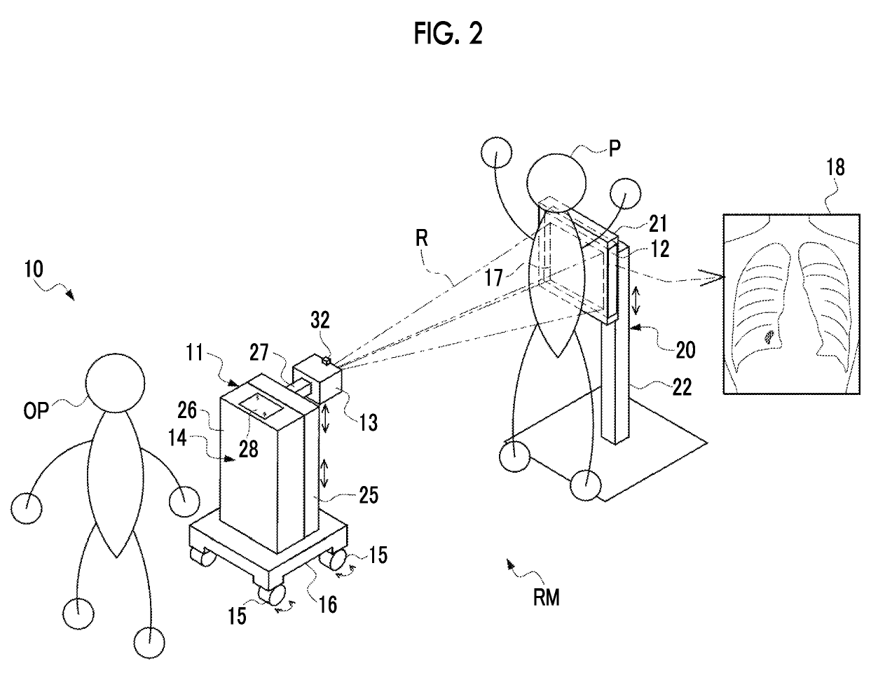

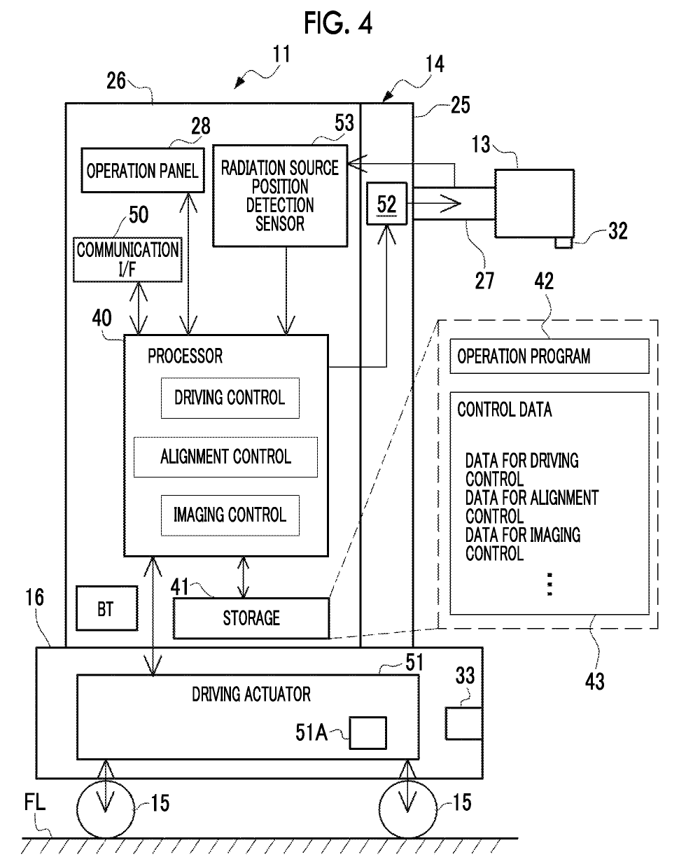

As shown in FIGS. 1 and 2 as an example, a radiography system 10 comprises a radiation generation apparatus 11 and an electronic cassette 12. The radiation generation apparatus 11 has a configuration in which a body part 14 including a radiation source 13 that emits radiation R toward a patient P is mounted on a carriage unit 16 including wheels 15. The radiation R is, for example, X-rays. A battery BT (see FIG. 4) is mounted in the radiation generation apparatus 11, and the radiation generation apparatus 11 can be driven by being supplied with power from the battery BT. In addition, the radiation generation apparatus 11 can also be driven while being supplied with power from a commercial power supply through a power supply cord. The radiation generation apparatus 11 is a device used for radiography and is an example of a "radiography apparatus" according to the disclosed technology. The carriage unit 16 is an example of a "driving mechanism" according to the disclosed technology. Further, the electronic cassette 12 is an example of a “radiographic image detection device” according to the technology of the present disclosure.

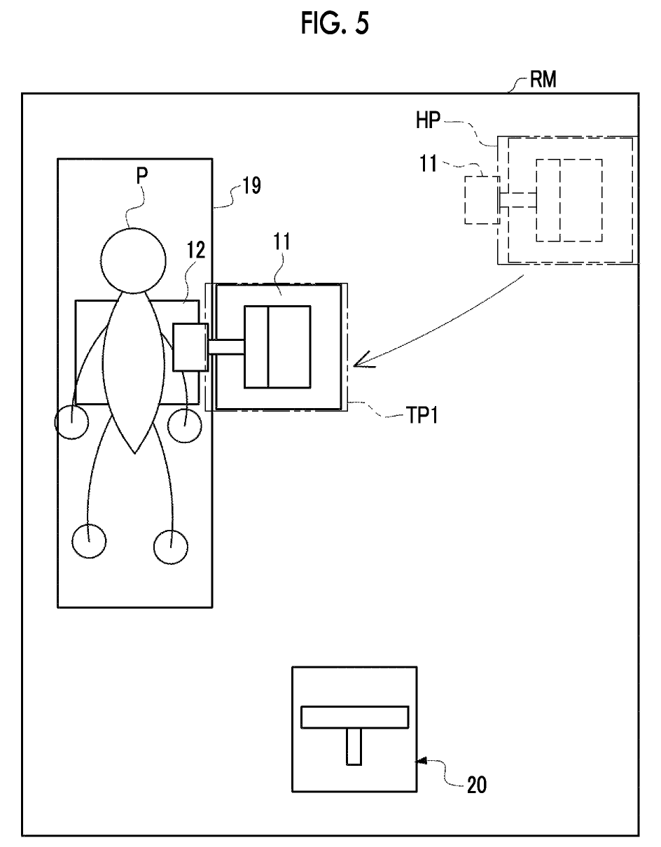

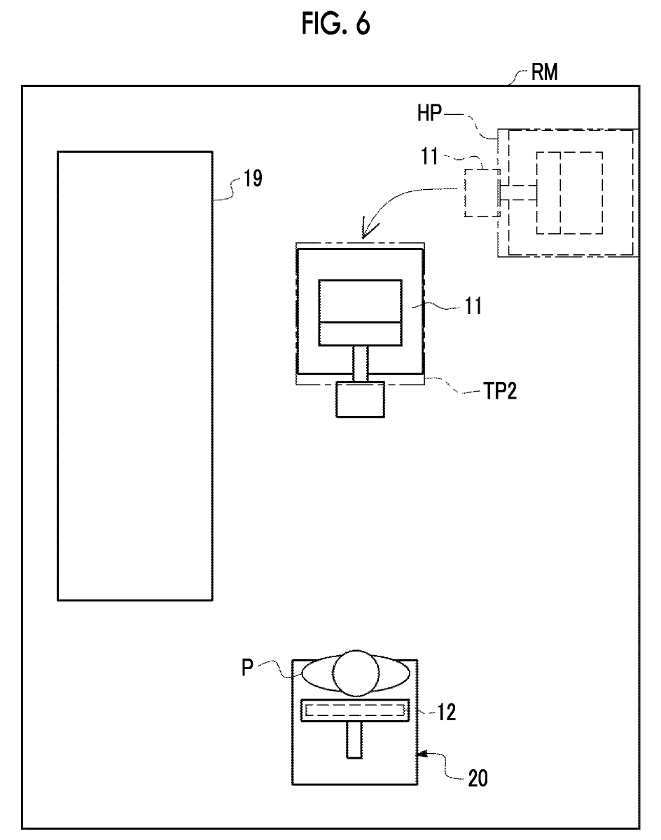

The radiation generation apparatus 11 can be moved in an imaging room RM (see also FIGS. 5 and 6). In addition, the radiation generation apparatus 11 is used for so-called ward round imaging in which a patient P is imaged while moving around a ward. Therefore, the radiation generation apparatus 11 is also called a ward round cart. Alternatively, the radiation generation apparatus 11 is also used for imaging in an emergency room. In addition, the radiation generation apparatus 11 can also be carried into an operating room and used during surgery.

The electronic cassette 12 has a configuration in which a sensor panel as a radiation detector that detects the radiation R is built in a portable housing. The electronic cassette 12 is driven by a battery and performs wireless communication with the radiation generation apparatus 11. The sensor panel has a detection surface 17 in which a plurality of pixels that generate signal charges in response to the radiation R or visible light converted from the radiation R by a scintillator are arranged in a matrix. The electronic cassette 12 detects the radiation R emitted from the radiation source 13 and transmitted through the patient P, and outputs a radiographic image 18 of the patient P.

Since the electronic cassette 12 is portable and wireless, as shown in FIG. 1, the electronic cassette 12 can be used for so-called free imaging in which the electronic cassette 12 is installed on a decubitus imaging table (bed) 19 installed in the imaging room RM (under the patient P) to perform radiography. More specifically, FIG. 1 shows a state in which the electronic cassette 12 is inserted between the decubitus imaging table 19 and the patient P to perform imaging for imaging the patient P lying on the decubitus imaging table 19 (see also FIG. 5). In addition, as shown in FIG. 2, the electronic cassette 12 can also be used by being accommodated in a holder 21 of an upright imaging table 20 installed in the imaging room RM (see also FIG. 6). The holder 21 can be raised and lowered in the up-down direction with respect to a support column 22.

The body part 14 has a rectangular-parallelepiped shape and is erected at the center of the carriage unit 16. The body part 14 is divided into a movable part 25 on a front side and a fixing unit 26 on a rear side. The movable part 25 can be raised and lowered in the up-down direction with respect to the fixing unit 26. The fixing unit 26 is fixed to the carriage unit 16.

A base end of an arm 27 is attached to the movable part 25. More specifically, the arm 27 is divided into a first portion in which the base end is attached to the movable part 25 and a second portion in which the base end is attached to the first portion. The radiation source 13 is attached to a distal end of the second portion, which is a free end opposite to the base end.

The first portion can be raised and lowered in the up-down direction with respect to the movable part 25 and can be rotated with respect to the movable part 25. The second portion can be bent in the up-down direction with respect to the first portion. Further, the second portion is extensible. The radiation source 13 can be rotated with respect to the second portion, that is, can be swung. By the displacement of the arm 27 and the displacement of the radiation source 13 with respect to the arm 27, a height position, a horizontal position, and a posture (orientation) of the radiation source 13 can be adjusted.

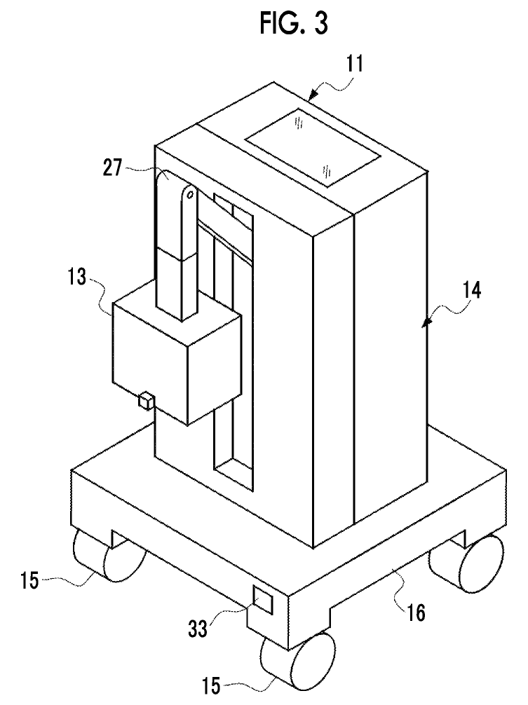

Since the second portion is bendable and extensible, the radiation source 13 can be moved to an imaging preparation position protruding toward the patient P and the electronic cassette 12 as shown in FIGS. 1 and 2 and an accommodation position pulled into the body part 14 as shown in FIG. 3 as an example. The imaging preparation position shown in FIGS. 1 and 2 is a position in a case in which the second portion is extended to a predetermined length, for example, a length of half of the longest length, and the second portion is bent with respect to the first portion such that the second portion is parallel to the horizontal direction. The accommodation position shown in FIG. 3 is a position in a case in which the second portion is shortened to the shortest and the second portion is folded as much as possible with respect to the first portion.

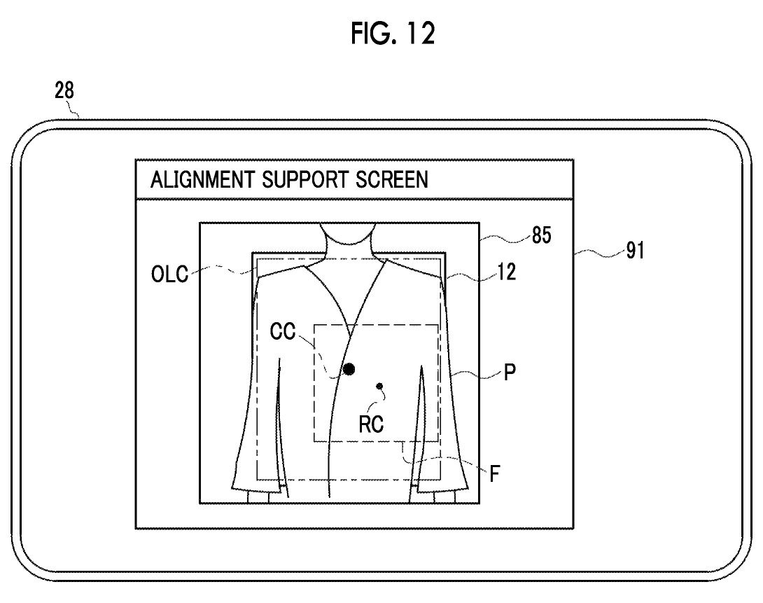

In FIGS. 1 and 2, an operation panel 28 is provided on an upper surface of the fixing unit 26. The operation panel 28 is configured by, for example, a touch panel display and has a function of displaying information in addition to an operation function. The operation panel 28 is operated by an operator OP such as a radiologic technologist. The operator OP sets an irradiation condition of the radiation R through the operation panel 28. In addition, the operator OP checks the radiographic image 18 through the operation panel 28. Further, as will be described later, the operator OP performs relative alignment (also referred to as positioning) between the radiation source 13 and the electronic cassette 12 with reference to an alignment support screen 91 (see FIG. 12) displayed on the operation panel 28.

In addition, an irradiation switch (not shown) is provided in the fixing unit 26. The irradiation switch is a switch that is provided to allow the operator OP to give an instruction to start irradiation of radiation. An extension cable is connected to the irradiation switch, and can be detached from the fixing unit 26 for use.

The radiation source 13 includes a radiation tube 30 and an irradiation field limiter 31. The radiation tube 30 generates the radiation R. The radiation tube 30 is provided with a filament, a target, a grid electrode, and the like (all are not illustrated). A tube voltage is applied between the filament that is a cathode and the target that is an anode from a voltage generator (not shown) built in the fixing unit 26. The filament releases thermal electrons according to the applied tube voltage toward the target. The target radiates the radiation R with collision of the thermoelectrons released from the filament. The grid electrode is disposed between the filament and the target, and changes a flow rate of the thermoelectrons from the filament toward the target in response to a voltage applied from the voltage generator. The flow rate of the thermal electrons from the filament toward the target is referred to as a tube current. The tube voltage and the tube current are set to the radiation source 13 as the irradiation condition with the irradiation time.

The irradiation field limiter 31 is also called a collimator or the like, and limits an irradiation field of the radiation R generated from the radiation tube 30. For example, the irradiation field limiter 31 has a configuration in which four shield plates formed of lead or the like shielding radiation R are disposed on respective sides of a quadrangle, and an emission opening of the quadrangle transmitting radiation is formed in a center portion. The irradiation field limiter 31 changes a size of the emission opening by changing a position of each shielding plate, thereby changing the irradiation field of the radiation R.

A camera 32 is attached to the radiation source 13. The camera 32 is used to support the alignment between the radiation source 13 and the electronic cassette 12. The camera 32 includes an imaging element that is sensitive to visible light, for example, a complementary metal oxide semiconductor (CMOS) image sensor or a charge-coupled device (CCD) image sensor. The camera 32 captures a video image. The camera 32 is an example of an "environment information sensor" according to the present disclosed technology.

The wheels 15 are provided in four positions in front, behind, left, and right of the carriage unit 16. That is, the carriage unit 16 is a four-wheel type. Each wheel 15 is, for example, a revolution type that revolves around a revolution axis extending in a height direction (also referred to as a vertical direction) orthogonal to a rotation axis in a case of driving and rotating. The radiation generation apparatus 11 autonomously drives by the carriage unit 16.

Here, the autonomous driving refers to autonomously driving toward a set target position while recognizing a surrounding environment using the environment information sensor and estimating a self-position. For example, a position on a side of the decubitus imaging table 19 shown in FIG. 1 and a position facing the upright imaging table 20 shown in FIG. 2 are preset as the target position. The radiation generation apparatus 11 uses a SLAM method using a SLAM technology as one of driving control methods for realizing the autonomous driving.

A camera 33 is provided in the carriage unit 16. The camera 33 is used to support the autonomous driving of the body part 14. The camera 33 images a front side of the carriage unit 16. The camera 33 also includes an imaging element that is sensitive to visible light and captures a video image, as in the camera 32. The camera 33 is also an example of an "environment information sensor" according to the disclosed technology, as in the camera 32. The body part 14 and the radiation generation apparatus 11 can be manually driven by the operator OP in addition to the autonomous driving.

FIG. 4 is a block diagram showing an example of an electric configuration of the radiation generation apparatus 11. A processor 40 integrally controls the entire radiation generation apparatus 11. The processor 40 is configured by, for example, a central processing unit (CPU) and a memory such as a random access memory (RAM), and functions as various processing units by loading and executing various programs in the memory. Specifically, the processor 40 executes driving control, alignment control, and imaging control. The driving control is control related to the autonomous driving of the carriage unit 16. The alignment control is control related to the alignment between the radiation source 13 and the electronic cassette 12 as described above. The imaging control includes irradiation control of the radiation R by the radiation source 13 and output control of the radiographic image 18 by the electronic cassette 12.

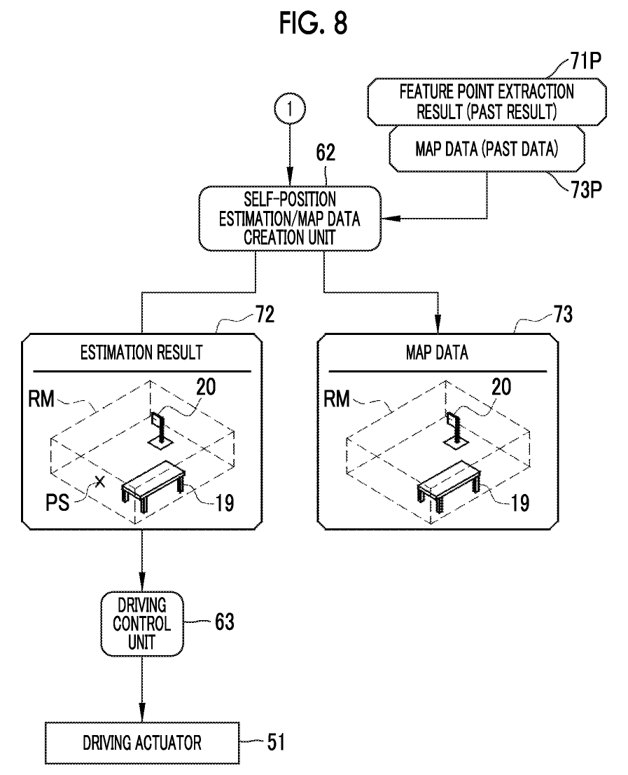

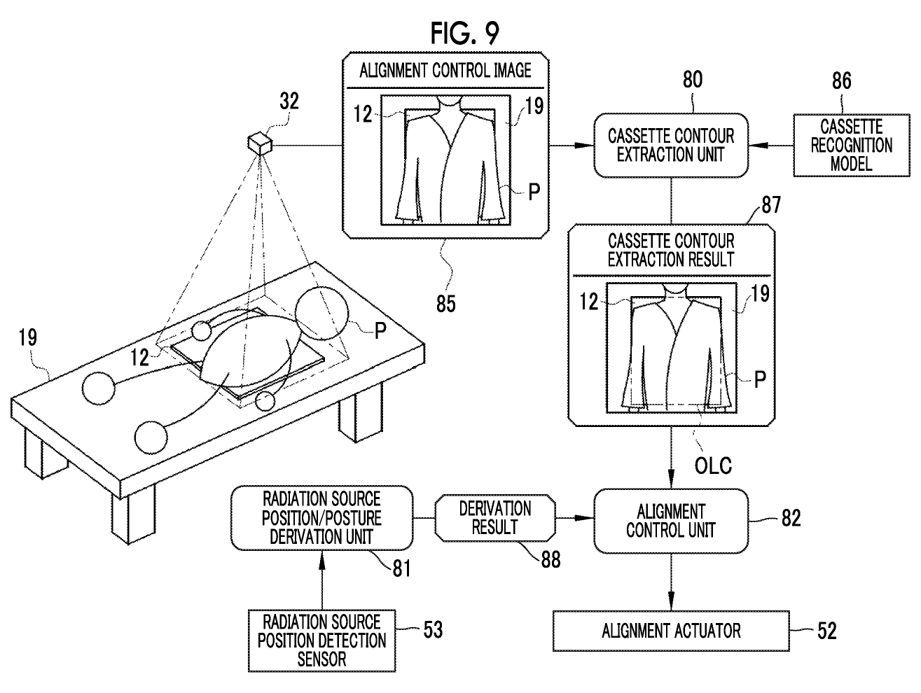

A storage 41 is configured by a non-volatile memory such as a hard disk drive or a solid state drive. The storage 41 stores an operation program 42 and control data 43. The operation program 42 is an example of an "operation program of a radiation generation apparatus" according to the disclosed technology. The control data 43 includes data for driving control, data for alignment control, and data for imaging control. Examples of the data for driving control include map data 73 (see FIG. 8) created in the SLAM method. Examples of the data for alignment control include a cassette recognition model 86 (see FIG. 9) for recognizing the electronic cassette 12. Examples of the data for imaging control include an irradiation condition table in which an irradiation condition for each imaging part is registered.

The operation panel 28, a communication interface (I/F) 50, a driving actuator 51, an alignment actuator 52, and a radiation source position detection sensor 53 are connected to the processor 40. The processor 40 performs display control of various screens on the operation panel 28. In addition, the processor 40 receives various operation instructions of the operator OP through the operation panel 28 and executes various controls corresponding to the various operation instructions. The communication I/F 50 is, for example, a wireless communication I/F and performs wireless communication with the electronic cassette 12.

The driving actuator 51 includes a motor for causing the wheels 15 to drive-rotate and a motor for causing the wheels 15 to revolve, under the control of the processor 40. In addition, the driving actuator 51 also includes a driving state detection sensor 51A that measures a rotation direction and a rotation amount of the wheels 15 and a revolution direction and a revolution angle. The driving state detection sensor 51A is, for example, a rotary encoder or a gyro sensor, or a combination of a plurality of types of these sensors. The driving state detection sensor 51A outputs the measurement value to the processor 40. The processor 40 detects the driving state such as the moving speed and the movement direction, in addition to the movement amount of the carriage unit 16, based on the measurement value from the driving state detection sensor.

The driving state can be used for various purposes. For example, the processor 40 can determine whether or not an operation state of the driving mechanism including the carriage unit 16 is normal based on the driving state detection sensor 51A.

In addition, the processor 40 uses the SLAM method as one of driving control methods for autonomous driving, but it is also possible to execute a driving control method other than the SLAM method by using the driving state detection sensor 51A. As will be described later, the SLAM method executes self-position estimation and creation of the map data based on the surrounding environment information to perform the autonomous driving. As a driving control method other than the SLAM method, for example, there is a method of executing the self-position estimation based on the movement amount detected by the driving state detection sensor 51A without creating the map data, and performing the autonomous driving. Of course, such a driving control method tends to have an increased error as the driving distance is longer as compared with the SLAM method, and thus is not suitable in a case in which the driving distance is long. However, it may be effective in a case in which the driving distance is short or the like, in which the method is used as an auxiliary.

The alignment actuator 52 includes a motor for causing the movable part 25 to be raised and lowered, a motor for causing the arm 27 to be raised and lowered, a motor for bending the second portion of the arm 27, a motor for extending and contracting the second portion, and a motor for rotating the radiation source 13 with respect to the second portion, under the control of the processor 40.

The radiation source position detection sensor 53 measures an elevation direction and an elevation amount of the arm 27 with respect to the movable part 25, a bending direction and a bending amount of the second portion of the arm 27 with respect to the first portion, an extension direction and an extension amount of the second portion, and a rotation direction and a rotation amount of the radiation source 13 with respect to the second portion. The radiation source position detection sensor 53 is, for example, a rotary encoder, a potentiometer, a gyro sensor, or a combination of a plurality of types of these sensors. The radiation source position detection sensor 53 outputs the measurement value to the processor 40. The processor 40 derives the position and the posture of the radiation source 13 based on the measurement value of the radiation source position detection sensor 53.

Since the radiation generation apparatus 11 can autonomously drive, the radiation generation apparatus 11 can automatically move to a designated position. As shown in FIGS. 5 and 6 as an example, a standby position HP of the radiation generation apparatus 11 is prepared in a corner of the imaging room RM. In the standby position HP, the radiation source 13 is in the accommodation position. In the standby position HP, charging of the battery BT, transmission of an imaging order from a radiology information system (RIS), setting of an irradiation condition, and the like are performed. The standby position HP occupies a region that is the same as or one size larger than the radiation generation apparatus 11.

In the imaging room RM, a first target position TP1 (see FIG. 5) of the radiation generation apparatus 11 in the decubitus imaging and a second target position TP2 (see FIG. 6) of the radiation generation apparatus 11 in the upright imaging are set. The first target position TP1 is a position on a side of the decubitus imaging table 19, and more specifically, a position facing a center portion of one long side of the decubitus imaging table 19. The second target position TP2 is a position facing the upright imaging table 20 at a distance of a source-to-image distance (SID) required for the upright imaging. In the following, the first target position TP1 and the second target position TP2 may be collectively referred to as a target position TP.

In a case of the decubitus imaging, the radiation generation apparatus 11 drives, for example, from the standby position HP toward the first target position TP1 and stops in a case in which the self-position PS (see FIG. 8) reaches the first target position TP1 (see FIG. 5). In addition, in a case of the upright imaging, the radiation generation apparatus 11 drives from the standby position HP toward the second target position TP2 and stops in a case in which the self-position PS reaches the second target position TP2. Although not shown, the radiation generation apparatus 11 can also drive from the first target position TP1 toward the second target position TP2 in order to perform the upright imaging after the decubitus imaging. In addition, the radiation generation apparatus 11 may drive from the second target position TP2 toward the first target position TP1 in order to perform the decubitus imaging after the upright imaging. In a case in which the autonomous driving to the target position TP is performed, the radiation source 13 is still in the accommodation position.

The autonomous driving to the target position TP is started, for example, by an instruction of the operator OP through the operation panel 28. In addition to or instead of the operation panel 28, a configuration may be adopted in which an instruction to start the autonomous driving can be issued by a remote controller.

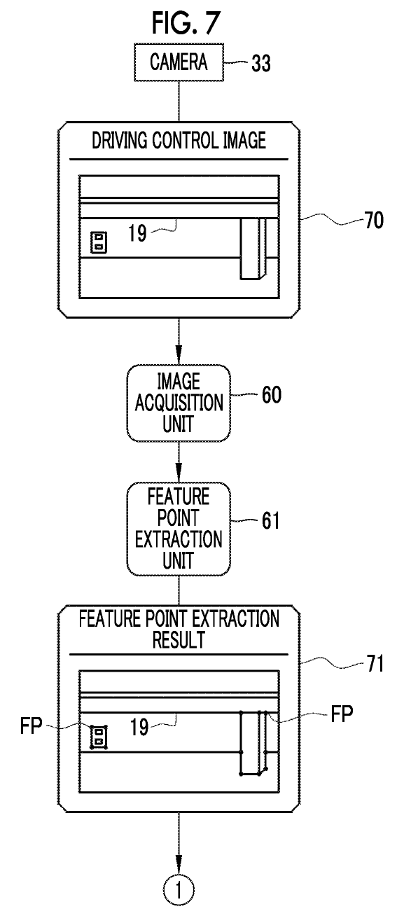

In the SLAM method, the self-position estimation is performed by continuously acquiring an image representing the surrounding environment and tracking the movement of the feature point in the image by image analysis, thereby realizing the autonomous driving. Specifically, as shown in FIGS. 7 and 8, the processor 40 functions as an image acquisition unit 60, a feature point extraction unit 61, a self-position estimation/map data creation unit 62, and a driving control unit 63 by the activation of the operation program 42.

The image acquisition unit 60 sequentially acquires an image 70 (hereinafter, referred to as a driving control image) of the surrounding environment of the radiation generation apparatus 11 captured by the camera 33. A frame rate, which is a frequency of acquiring the driving control image 70, is controlled according to a driving state of the driving mechanism of the radiation generation apparatus 11 as will be described below. The image acquisition unit 60 performs preprocessing such as noise removal and distortion correction on the driving control image 70, and then outputs the driving control image 70 to the feature point extraction unit 61.

The feature point extraction unit 61 extracts a corner of a structure present in the surrounding environment shown in the driving control image 70 as a feature point FP by using an algorithm such as oriented features from accelerated segment test and rotated binary robust independent elementary features (ORB) or speeded-up robust features (SURF). The feature point extraction unit 61 outputs a feature point extraction result 71 of the feature point FP to the self-position estimation/map data creation unit 62. In addition, although not shown, the feature point extraction unit 61 stores the feature point extraction result 71 in the storage 41. The feature point extraction result 71 is a set of coordinates and a feature amount vector of each feature point FP.

The self-position estimation/map data creation unit 62 estimates the self-position PS of the radiation generation apparatus 11 and creates the map data 73 of the surrounding environment. The feature point extraction result 71 is input to the self-position estimation/map data creation unit 62 from the feature point extraction unit 61. In addition, a feature point extraction result (hereinafter, referred to as an extraction result (past result)) 71P for a plurality of past frames and map data (hereinafter, referred to as map data (past data)) 73P for a plurality of past frames are input to the self-position estimation/map data creation unit 62.

The feature point extraction result (past result) 71P and the map data (past data) 73P are stored in the storage 41 as the data for driving control of the control data 43. The feature point extraction result 71 and the map data 73 constituting the feature point extraction result (past result) 71P and the map data (past data) 73P are, for example, for several tens to several hundreds of frames. The feature point extraction result (past result) 71P and the map data (past data) 73P include the feature point extraction result 71 and the map data 73 that are considered to play an important role in the estimation of the self-position PS and the creation of the map data 73. The feature point extraction result 71 and the map data 73 are, for example, the feature point extraction result 71 and the map data 73 obtained for each movement of a certain distance. In addition, for example, the feature point extraction result 71 and the map data 73 obtained in a case in which a large viewpoint change has occurred from the previous frame. Further, for example, the feature point extraction result 71 and the map data 73 obtained in a case in which a set amount or more of new feature points FP are extracted.

The self-position estimation/map data creation unit 62 collates the feature point FP of the feature point extraction result 71 from the feature point extraction unit 61 with the feature point FP of the feature point extraction result (past result) 71P. In this case, the self-position estimation/map data creation unit 62 refers to the feature amount vector of each feature point FP. More specifically, the feature point extraction unit 61 recognizes the feature points FP having a distance (Euclidean distance or the like) of the feature amount vector less than the threshold value between the feature point FP of the feature point extraction result 71 from the feature point extraction unit 61 and the feature point FP of the feature point extraction result (past result) 71P as the same feature point FP. The self-position estimation/map data creation unit 62 estimates the self-position PS based on the collation result of the feature point FP and the map data (past data) 73P. In addition, the self-position estimation/map data creation unit 62 creates (updates the map data 73) the new map data 73 based on the feature point extraction result 71 from the feature point extraction unit 61, the feature point extraction result (past result) 71P, the map data (past data) 73P, and an estimation result 72. As described above, the self-position estimation/map data creation unit 62 estimates the self-position PS and creates the map data 73 in parallel in a process in which the radiation generation apparatus 11 autonomously drives.

The self-position estimation/map data creation unit 62 outputs the estimation result 72 of the self-position PS to the driving control unit 63. The driving control unit 63 controls the driving of the driving actuator 51 such that the self-position PS is the target position TP. In addition, although not shown, the self-position estimation/map data creation unit 62 stores the map data 73 in the storage 41. Specifically, the map data 73 is three-dimensional data of the imaging room RM including a structure such as the decubitus imaging table 19 and the upright imaging table 20. In addition, the standby position HP and the target position TP are registered in the map data 73.

As shown in FIG. 9 as an example, the processor 40 functions as a cassette contour extraction unit 80, a radiation source position/posture derivation unit 81, and an alignment control unit 82.

An image 85 (hereinafter, referred to as an alignment control image) including the patient P and the electronic cassette 12 captured by the camera 32 is sequentially input to the cassette contour extraction unit 80. The camera 32 is attached to the radiation source 13. Therefore, in a case in which the self-position PS of the radiation generation apparatus 11 is the target position TP and the radiation source 13 is the imaging preparation position, the patient P and the electronic cassette 12 are shown in the alignment control image 85. In FIG. 9, since the decubitus imaging is shown as an example, the decubitus imaging table 19 is also shown in the alignment control image 85. It is assumed that the alignment between the patient P and the electronic cassette 12 is completed by the operator OP before the alignment control.

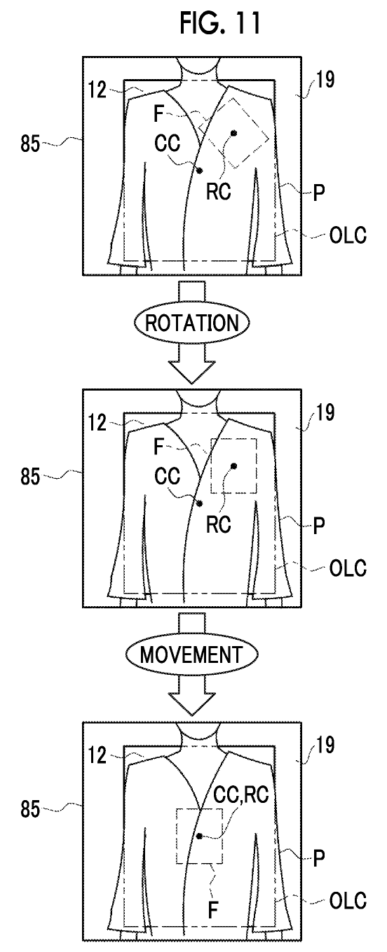

The cassette contour extraction unit 80 extracts a contour OLC of the electronic cassette 12 from the alignment control image 85 by using the cassette recognition model 86. According to the contour OLC, a center CC (see FIG. 11) of the detection surface of the electronic cassette 12 is known. The cassette contour extraction unit 80 outputs a cassette contour extraction result 87 to the alignment control unit 82. Here, the contour following all sides of the electronic cassette 12 is shown as the contour OLC, but the present disclosure is not limited to this. Four corners of the electronic cassette 12 may be extracted as the contour. The alignment control image 85 is an example of "output data" according to the disclosed technology.

The cassette recognition model 86 is a trained model that is configured by, for example, a convolutional neural network or the like and that has been trained to output the cassette contour extraction result 87 in a case in which the alignment control image 85 is input. The cassette recognition model 86 is stored in the storage 41 as the data for alignment control of the control data 43.

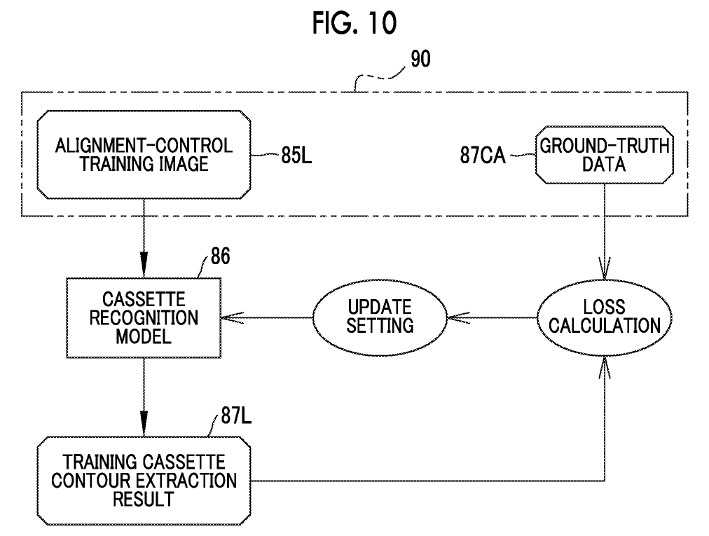

As shown in FIG. 10 as an example, in the learning phase, learning data 90 is given to the cassette recognition model 86. The learning data 90 is composed of a set of an alignment-control training image 85L and ground-truth data 87CA. The electronic cassette 12 is shown in the alignment-control training image 85L. The ground-truth data 87CA is data in which the contour OLC of the electronic cassette 12 shown in the alignment-control training image 85L is annotated, and is data for checking the answer.

The alignment-control training image 85L is input to the cassette recognition model 86. The cassette recognition model 86 outputs a training cassette contour extraction result 87L in response to the input of the alignment-control training image 85L. The loss calculation of the cassette recognition model 86 using the loss function is performed based on the training cassette contour extraction result 87L and the ground-truth data 87CA. Then, the update setting of various coefficients (coefficients of a filter of a convolutional layer and the like) of the cassette recognition model 86 is made according to the result of the loss calculation, and the cassette recognition model 86 is updated according to the update setting.

In the learning phase of the cassette recognition model 86, the series of processing of the input of the alignment-control training image 85L to the cassette recognition model 86, the output of the training cassette contour extraction result 87L from the cassette recognition model 86, the loss calculation, the update setting, and the update of the cassette recognition model 86 is repeatedly performed while the learning data 90 is replaced. The repetition of the series of processing is ended in a case in which the extraction accuracy of the training cassette contour extraction result 87L reaches a predetermined set level. The cassette recognition model 86 in which the extraction accuracy has reached the set level is stored in the storage 41 and is used by the cassette contour extraction unit 80. It should be noted that the learning may be ended in a case in which the series of processing is repeated a set number of times regardless of the extraction accuracy of the training cassette contour extraction result 87L.

The radiation source position/posture derivation unit 81 derives the position and the posture of the radiation source 13 based on the measurement value of the radiation source position detection sensor 53. The radiation source position/posture derivation unit 81 outputs a derivation result 88 to the alignment control unit 82. The derivation result 88 is coordinates of an irradiation center RC (see FIG. 11) of the radiation R in the alignment control image 85 and coordinates of a rectangular frame F (see FIG. 11) indicating the posture of the radiation source 13 in the alignment control image 85. Since the positional relationship between the radiation source 13 and the camera 32 is known, the coordinates of the irradiation center RC and the frame F in the alignment control image 85 can be easily converted from the measurement value of the radiation source position detection sensor 53.

The alignment control unit 82 controls the driving of the alignment actuator 52 such that the radiation source 13 and the electronic cassette 12 face each other. More specifically, as shown in FIG. 11 as an example, in a case in which the posture of the radiation source 13 is inclined with respect to the electronic cassette 12, the alignment control unit 82 rotates the radiation source 13 to eliminate the inclination. In addition, in a case in which the irradiation center RC of the radiation R and the center CC of the detection surface of the electronic cassette 12 are shifted, the alignment control unit 82 moves the radiation source 13 to eliminate the shift. It should be noted that the inclination may be eliminated after the shift between the irradiation center RC of the radiation R and the center CC of the detection surface of the electronic cassette 12 is eliminated. In addition, in FIG. 11, a case in which the posture of the radiation source 13 is inclined around a normal line of the detection surface of the electronic cassette 12 is shown as an example, but the present disclosure is not limited to this. Even in a case in which the posture of the radiation source 13 is inclined around an axis along the long side of the detection surface of the electronic cassette 12 or around an axis along the short side of the detection surface of the electronic cassette 12, the alignment control unit 82 rotates the radiation source 13 to eliminate the inclination.

As shown in FIG. 12, the alignment control unit 82 displays an alignment support screen 91 showing a state of the alignment between the radiation source 13 and the electronic cassette 12 on the operation panel 28. The alignment control image 85 is displayed on the alignment support screen 91. The operator OP can check the state of the alignment between the radiation source 13 and the electronic cassette 12 through the alignment support screen 91.

In addition, although the example in which the alignment of the radiation source 13 is automatically performed by the alignment control unit 82 has been described, the operator OP may manually perform the alignment of the radiation source 13. The manual operation may be, for example, an operation in which the operator OP inputs an operation instruction to the alignment actuator 52 through an operation button or the like, or an operation in which the operator OP directly moves the radiation source 13. Of course, in a case in which the radiation source 13 is displaced by the manual operation of the operator OP, the alignment control image 85 in the alignment support screen 91 is updated. As a result, the operator OP can check the current state of the alignment through the alignment support screen 91.

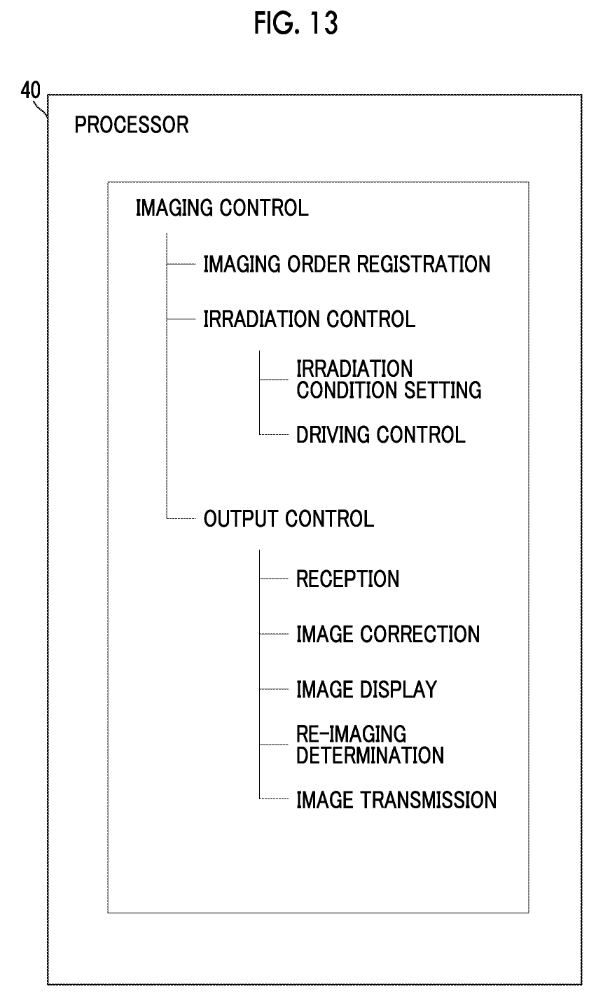

As shown in FIG. 13, the processor 40 further executes the imaging control. The imaging control includes, for example, control such as imaging order registration, irradiation control, and output control. The imaging order registration is processing of registering information such as patient information, an imaging purpose, and an imaging part based on an imaging order received from the RIS or the like.

The irradiation control includes irradiation condition setting and driving control. The irradiation condition setting is setting of an irradiation condition including a tube voltage, a tube current, and an irradiation time of the radiation generated by the radiation source 13. The driving control is driving control of the radiation source 13 according to the set irradiation condition, and the driving control includes synchronization control of an irradiation timing of the radiation source 13 and an image detection timing of the electronic cassette 12.

The output control includes processing of receiving the radiographic image 18 detected by the electronic cassette 12, image correction of performing various types of correction such as offset correction, sensitivity correction, and defect correction on the received radiographic image 18, image display of displaying the corrected radiographic image 18 on the operation panel 28, and the like. Further, the output control includes re-imaging determination and image transmission. The re-imaging determination is processing of determining whether or not the imaged radiographic image 18 can be used for diagnosis in light of the imaging purpose, and determining whether or not re-imaging is necessary. The image transmission is processing of transmitting the imaged radiographic image 18 to an image server.

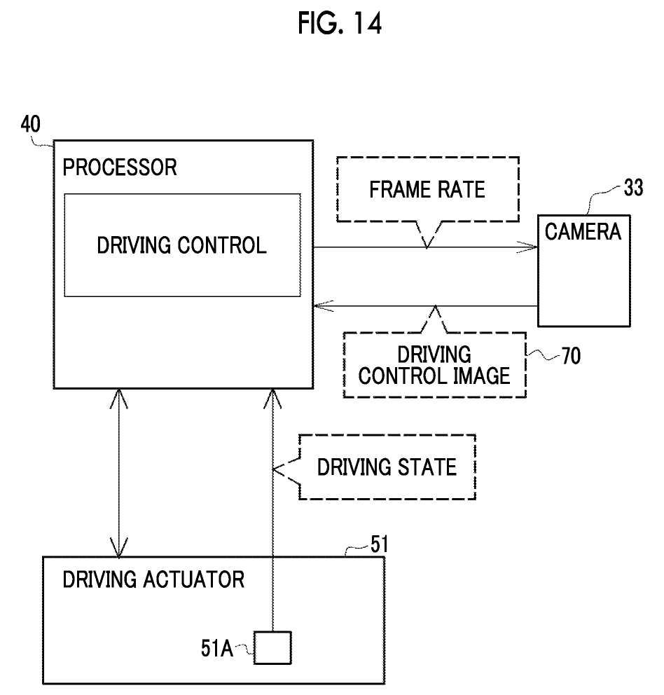

Further, as shown in FIG. 14, the processor 40 has a function of controlling the frame rate of the camera 33 according to the driving state of the driving mechanism including the carriage unit 16. The camera 33 is a camera that images the surrounding environment, and the frame rate is the acquisition frequency of the driving control image 70 captured by the camera 33. Here, the driving control image 70 is an example of "surrounding environment information" according to the present disclosed technology, and the camera 33 is an example of an "environment information sensor" according to the present disclosed technology. Further, the frame rate is an example of "information acquisition frequency" according to the present disclosed technology.

The processor 40 transmits the frame rate to the camera 33 as an operation condition according to the driving state acquired from the driving state detection sensor 51A. In a case in which the frame rate is received, the camera 33 captures the video image at the received frame rate. The driving state includes a movement direction, a movement amount, and the like. More specifically, the driving state includes straight movement in which the movement direction does not change and rotational movement in which the movement direction changes, and the straight movement further includes forward and backward movement, lateral movement, and diagonal movement. The forward and backward movement is linear movement along the front-rear direction of the carriage unit 16, and the lateral movement is linear movement along the left-right direction of the carriage unit 16 orthogonal to the front-rear direction. The diagonal movement is, for example, straight movement along a direction between the front-rear direction and the lateral movement, such as 45 degrees diagonally in a case in which the front-rear direction is 0° and the left-right direction is 90°. The reason for controlling the frame rate according to such a driving state is as follows.

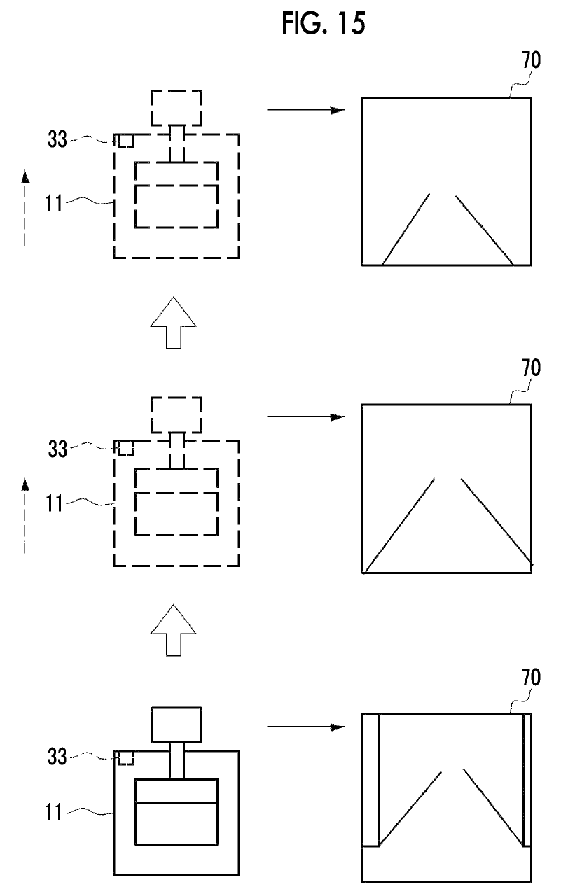

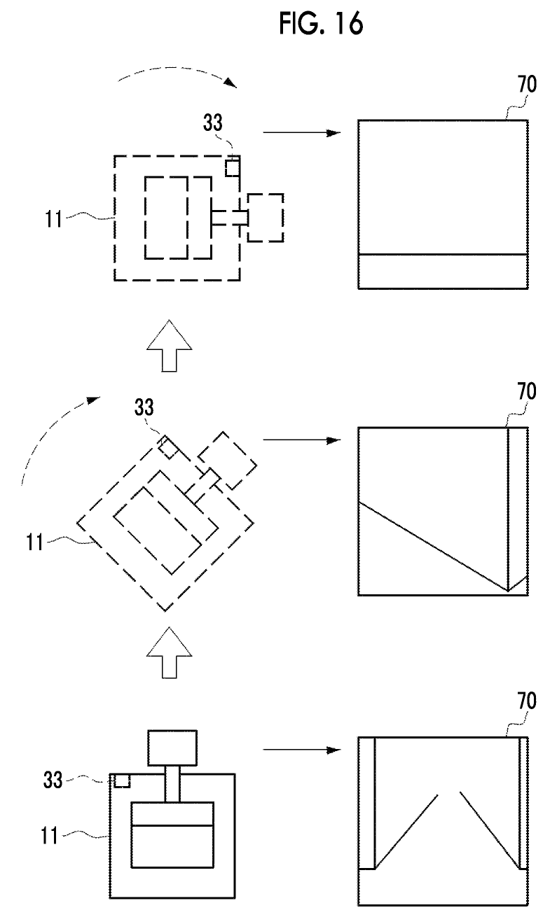

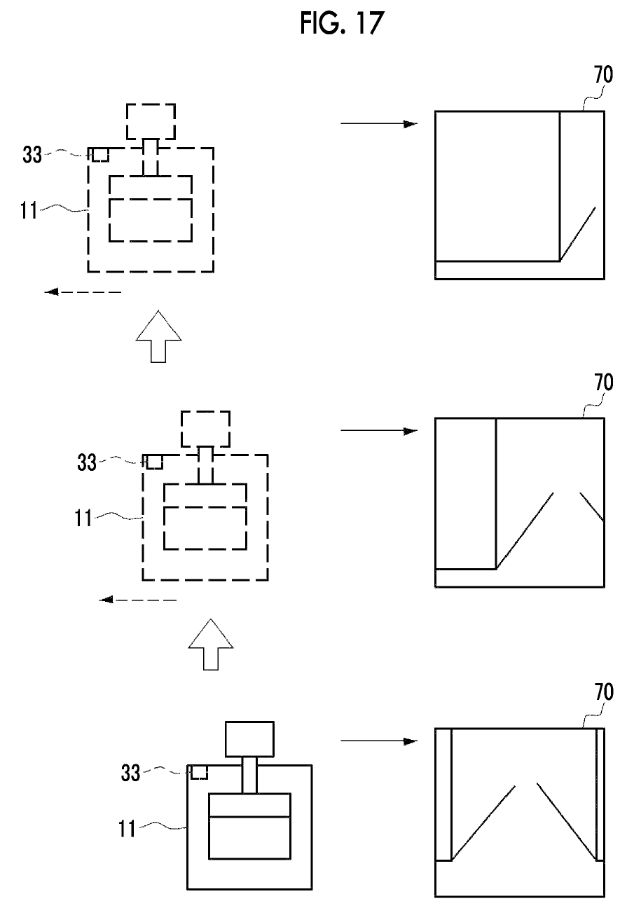

FIGS. 15 to 17 schematically show a state of a change in the driving control images 70 sequentially acquired in a case in which the radiation generation apparatus 11 including the carriage unit 16 is moved. FIG. 15 is an example of the straight movement, FIG. 16 is an example of the rotational movement, and FIG. 17 is an example of the lateral movement. In FIGS. 15 to 17, it is assumed that a straight corridor in which both sides are walls extends in front of the radiation generation apparatus 11 in an initial state (the lowermost in each of FIGS. 15 to 17) of the radiation generation apparatus 11. FIGS. 15 to 17 show how each driving control image 70 continuously acquired by the camera 33 changes according to the driving state from the initial state of the radiation generation apparatus 11.

As shown in FIG. 15, in a case in which the radiation generation apparatus 11 moves straight forward (that is, moves forward) from the initial state, the radiation generation apparatus 11 proceeds through the straight corridor. In this case, since the viewpoint of the camera 33 moves only along the direction in which the corridor extends, it is considered that the change amount of the driving control images 70 continuously acquired by the camera 33 is relatively small. On the other hand, as shown in FIG. 16, in a case in which the radiation generation apparatus 11 rotates from the initial state to change the direction, the orientation of the viewpoint of the camera 33 that images the corridor changes, and thus it is considered that the change amount of the driving control images 70 continuously acquired by the camera 33 is larger than that in the case of moving forward shown in FIG. 15. In addition, as shown in FIG. 17, in a case in which the radiation generation apparatus 11 laterally moves from the initial state, the viewpoint of the camera 33 moves in the lateral direction orthogonal to the direction in which the corridor extends, and thus it is considered that the change amount of the driving control images 70 continuously acquired by the camera 33 is larger than that in the case of moving forward shown in FIG. 15.

As described above, in the SLAM method, the processor 40 extracts the feature point FP from each of the plurality of driving control images 70 continuously acquired, and performs the self-position estimation (including the update of the map data 73) by tracking the movement of the feature point FP. Therefore, in a case in which the frame rate is always constant regardless of each driving state shown in FIGS. 15 to 17, the accuracy of the self-position estimation may be decreased.

This is because, in the case of the rotational movement as shown in FIG. 16, the change amount of the image is relatively large as compared with the straight movement. Therefore, the movement amount of the feature point FP (see FIGS. 7 and 8) in the plurality of images continuously acquired as the driving control images 70 is larger as the frame rate is lower (that is, the image acquisition interval is longer). In a case in which the movement amount of the feature point FP is large, the search time is long, and in a case in which the feature point FP is not found within a specified time, the self-position estimation cannot be accurately performed, and thus the estimation accuracy is decreased. On the other hand, in the case of the straight movement in the front-rear direction as shown in FIG. 15, in a case in which the frame rate is the same as in the case shown in FIG. 16, the change amount of the plurality of images continuously acquired as the driving control images 70 is small. Therefore, even in a case in which the frame rate is low (that is, the image acquisition interval is long), the search time of the feature point FP is short as compared with the rotational movement shown in FIG. 16. The change amount of the image in the case of the lateral movement as shown in FIG. 17 is considered to be an intermediate change amount between the forward and backward movement shown in FIG. 15 and the rotational movement shown in FIG. 16. In addition, although not shown, the straight movement also includes the diagonal movement as described above, in addition to the forward and backward movement and the lateral movement. The change amount of the image in the diagonal movement is also considered to be an intermediate change amount as in the lateral movement.

However, the frame rate is not always higher is better, and there is a disadvantage in increasing the frame rate. For example, in a case in which the frame rate is too high in a case in which the change amount of the image is small as in the forward and backward movement shown in FIG. 15, the movement amount of the feature point FP is too small, and the movement is erroneously determined to be not moved. In this case, it is likely that the erroneous determination can be suppressed by decreasing the frame rate to some extent to increase the movement amount of the feature point FP.

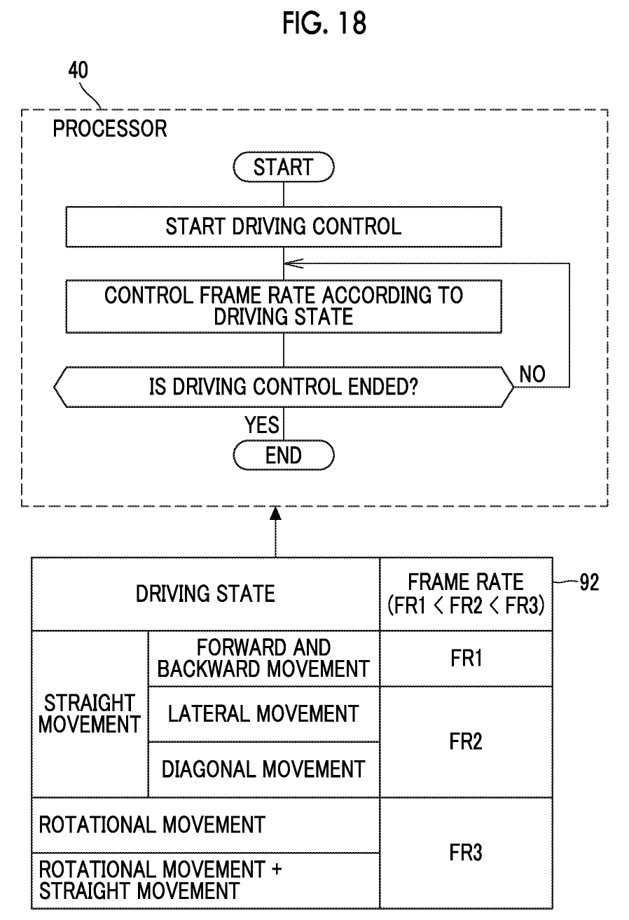

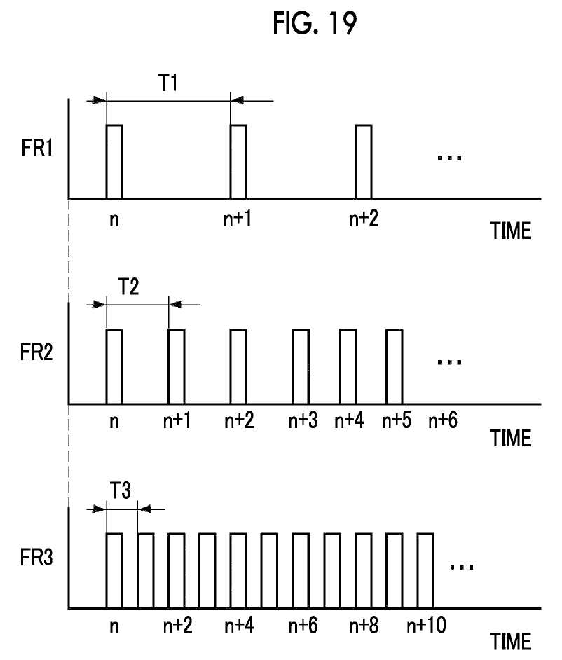

Therefore, in the radiation generation apparatus 11, the processor 40 controls the frame rate according to the driving state based on, for example, a table 92 shown in FIG. 18. In the table 92, FR1, FR2, and FR3 are values of the frame rates. As also shown in FIG. 19, the image acquisition intervals indicated by T1 to T3 are shorter as the frame rate is higher. Among FR1 to FR3, FR3 is the highest, FR1 is the lowest, and FR2 is intermediate. In the table 92, in a case of the forward and backward movement in the straight movement, the lowest FR1 is set, and in a case of the rotational movement, the highest FR3 is set. In the straight movement, the lateral movement and the diagonal movement other than the forward and backward movement are set to the intermediate FR2. In addition, in a case in which the rotational movement and the straight movement are performed in parallel, the same FR3 as the rotational movement is set. As specific values of FR1 to FR3, for example, FR1 is 1 frames per second (FPS), FR3 is 4 FPS, and FR2 is 2 FPS, which is intermediate. In a case in which FR1 is a reference, FR2 is a value twice as large as FR1, and FR3 is a value four times as large as FR1.

As shown in a flowchart of FIG. 18, in a case in which the driving control is started, the processor 40 controls the frame rate according to the driving state while referring to the table 92. Then, the control of the frame rate is continued until the driving control is ended. As a result, in the radiation generation apparatus 11, the driving control image 70 is acquired at an appropriate interval according to the driving state, and thus the accuracy of the self-position estimation is improved.

As described above, the radiation generation apparatus 11, which is an example of the radiography apparatus according to the present disclosed technology, comprises the processor 40 that controls the driving mechanism including the carriage unit 16 as an example, and the processor 40 controls the frame rate (an example of the information acquisition frequency) of the camera 33 (an example of the environment information sensor) according to the driving state of the driving mechanism. As a result, the driving control of the autonomous driving can be appropriately performed as compared with the related art.

The present disclosed technology is particularly effective in the radiography apparatus in which the radiation generation apparatus 11 is shown as an example as described below. That is, the SLAM method is also used for, for example, a transport robot of a cargo in a warehouse. In such an application in the warehouse, in response to the decrease in the accuracy of the self-position estimation as described above, a marker such as a line marker may be provided on a moving path of the driving mechanism. That is, in a case in which a marker such as a line marker is provided on the moving path of the driving mechanism, the line marker shown in the image is a clue in a case in which the self-position estimation is executed, and it is considered that the estimation accuracy is not easily decreased even in a case in which the frame rate is always constant. In addition, the moving path of the driving mechanism in the warehouse is often secured in a certain order, and it is considered that the accuracy of the self-position estimation is easily ensured by such an environmental factor.

On the other hand, in an environment such as the imaging room RM in which the radiography apparatus is used, various medical apparatuses are disposed on the moving path of the driving mechanism, and thus it is difficult to provide a marker such as a line marker. Further, in the imaging room RM or the like, the layout of the medical apparatus also changes every day, and it is often difficult to secure a certain moving path in an orderly manner. Therefore, in a case in which the SLAM method is used in the radiography apparatus, it is difficult to adopt a measure of using the line marker as a measure against the decrease in the accuracy of the self-position estimation.

The present disclosed technology is particularly effective in the radiography apparatus because the decrease in the accuracy of the self-position estimation can be suppressed by appropriately controlling the frame rate even in an environment such as the imaging room RM in which the line marker is not provided.

In addition, the radiation generation apparatus 11 distinguishes between the straight movement in which the movement direction does not change and the rotational movement in which the movement direction changes for the driving state. Further, the straight movement is distinguished into the forward and backward movement, the lateral movement, and the diagonal movement. As a result, it is possible to set an appropriate frame rate (an example of the information acquisition frequency) according to the distinction of each driving state, and thus it is possible to improve the accuracy of the self-position estimation as compared with a case in which such a distinction is not made.

Specifically, as shown in the table 92 of FIG. 18 as an example, the processor 40 sets the frame rate (FR3) of the rotational movement to be higher than the frame rate (FR1) of the straight movement. In addition, the processor 40 sets the frame rate (FR2) of the lateral movement or the diagonal movement to be higher than the frame rate (FR1) of the forward and backward movement. As described above, it is considered that the change amount of the plurality of images continuously acquired is large in the order of the forward and backward movement, the lateral movement or the diagonal movement, and the rotational movement, and thus it is considered to be appropriate to increase the frame rate in this order in order to improve the accuracy of the self-position estimation. In addition, in the table 92, the frame rate (FR3) in a case in which the rotational movement and the straight movement are performed in parallel is higher than the frame rate (FR1 and FR2) in a case in which only the straight movement is performed. In a case of including the rotational movement, the change amount of the image is large, and thus such a setting of the frame rate is also considered to be appropriate.

### Modification Example 1: Control of Frame Rate according to Moving Speed

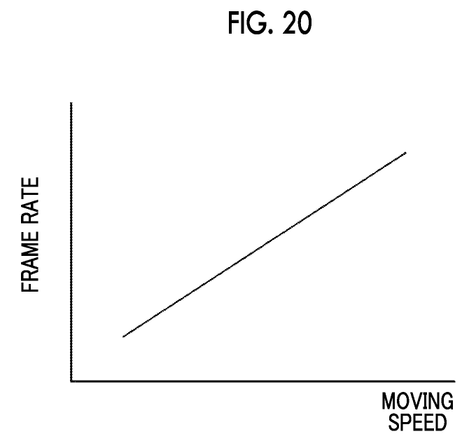

In addition, as shown in FIG. 20, the processor 40 may set the frame rate to be higher as the moving speed of the driving mechanism is faster. Of course, the movement amount of the driving mechanism per unit time is larger as the moving speed is faster. In this case, in a case in which the frame rate is increased to shorten the acquisition interval of the driving control image 70, it is possible to suppress the increase in the change amount between the plurality of images continuously acquired, and it is possible to improve the accuracy of the self-position estimation.

### Modification Example 2: Control of Frame Rate according to Movement Direction and Moving Speed

In addition, the control of the frame rate according to the moving speed may be combined with the movement direction. That is, the frame rate may be controlled by considering both the moving speed and the movement direction. In this manner, the frame rate can be more appropriately controlled according to the driving state.

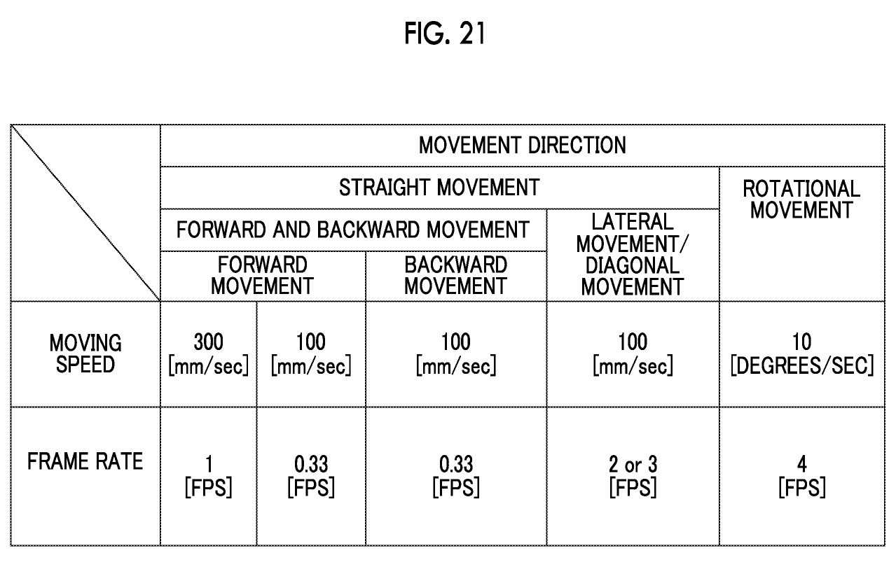

FIG. 21 is an example of a setting content of the frame rate according to both the movement direction and the moving speed. In FIG. 21, as described above, the movement direction is first distinguished between the straight movement and the rotational movement, and the straight movement is distinguished into the forward and backward movement, the lateral movement, and the diagonal movement. In addition, the forward and backward movement is distinguished between the forward movement and the backward movement. In each movement direction, there is a moving speed of the driving mechanism of the radiation generation apparatus 11 assumed, and in the example of FIG. 21, the straight movement other than the forward movement is assumed to be 100 [mm/sec]. In addition, the rotational movement is assumed to be 10 [degrees/sec]. The frame rate is set in each movement direction on the premise of such a moving speed. Specifically, 0.33 FPS is set for the backward movement, and 2 FPS or 3 FPS is set for the lateral movement/diagonal movement. In a case of the rotational movement, it is 4 FPS. The values of these frame rates are higher as the change amount between the images is larger, and are based on the same idea as in the example of FIG. 18.

In addition, in the example shown in FIG. 21, the frame rate is controlled by considering not only the movement direction but also the moving speed for the forward movement. That is, even in the case of the forward movement, two types of the moving speed of 300 [mm/sec] and the moving speed of 100 [mm/sec] are assumed, and the frame rate is controlled according to each moving speed. Specifically, in a case in which the moving speed is 300 [mm/sec], the frame rate is set to 1 FPS, and in a case in which the moving speed is 100 [mm/sec], the frame rate is set to 0.33 FPS, and thus the frame rate is set to be low in a case in which the moving speed is slow even in the forward movement in which the movement direction is the same.

One of the reasons why two types of moving speeds are assumed in the forward movement is as follows. For example, in a case in which the radiation generation apparatus 11 is moved from the standby position HP shown in FIGS. 5 and 6 to each target position TP of the first target position TP1 or the second target position TP2, the movement to the vicinity of the target position TP is performed at a relatively fast moving speed, and in a case in which the target position TP is approached, the moving speed is decreased to perform the final position adjustment to the target position TP. In this case, it is considered to decrease the moving speed even in the forward movement as a fine adjustment mode in which the final position adjustment is performed. In this case, since the moving speed is slow, it is appropriate from the viewpoint of improving the accuracy of the self-position estimation described above to set the frame rate to be lower by that amount. As described above, the frame rate may be controlled by considering both the movement direction and the moving speed. FIG. 21 is an example, and the frame rate may be set according to both the movement direction and the moving speed for the movement other than the forward movement.

### Modification Example 3

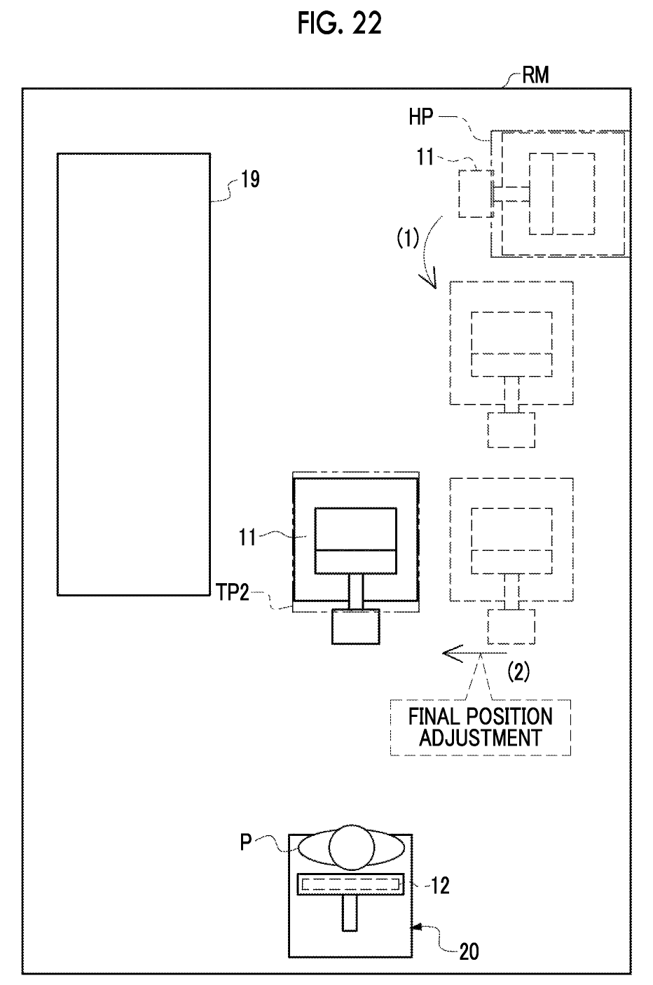

In addition, as shown in FIG. 22, the processor 40 may execute the driving control such that the final position adjustment to the target position TP is the straight movement. As shown in FIG. 22, in a case in which the radiation generation apparatus 11 is moved from the standby position HP to the target position TP (in FIG. 22, the second target position TP2), immediately after the movement is started from the standby position HP, as shown in (1), the rotational movement such as the direction change is performed. In a case in which the radiation generation apparatus 11 approaches the target position TP, specifically, in a case in which the radiation generation apparatus 11 enters a proximity range of the target position TP set in advance, the radiation generation apparatus 11 executes the final position adjustment as shown in (2). The processor 40 causes the radiation generation apparatus 11 to complete the direction change to the posture facing the electronic cassette 12, for example, during the period from the standby position HP to the target position TP. As a result, the final position adjustment can be the straight movement. The accuracy of the self-position estimation is higher in the straight movement than in the rotational movement. Therefore, by executing the final position adjustment by the straight movement having high accuracy, it is possible to accurately perform the alignment of the radiation generation apparatus 11 and the electronic cassette 12. Further, it is preferable that the final position adjustment is completed only by the straight movement such that the final position adjustment does not include a slight rotational movement. As described above, such an operation procedure may be devised in addition to the control of the frame rate according to the driving state.

In addition, as a method of determining whether or not the radiation generation apparatus 11 approaches the target position TP, for example, the following method can be considered in addition to a case in which the difference between the target position TP set in advance and the estimated self-position is within a predetermined range. One is that the electronic cassette 12 or the patient P is detected based on the driving control image 70. It is not only detected, but may be determined by considering the distance to the electronic cassette 12 or the patient P.

### Modification Example 4

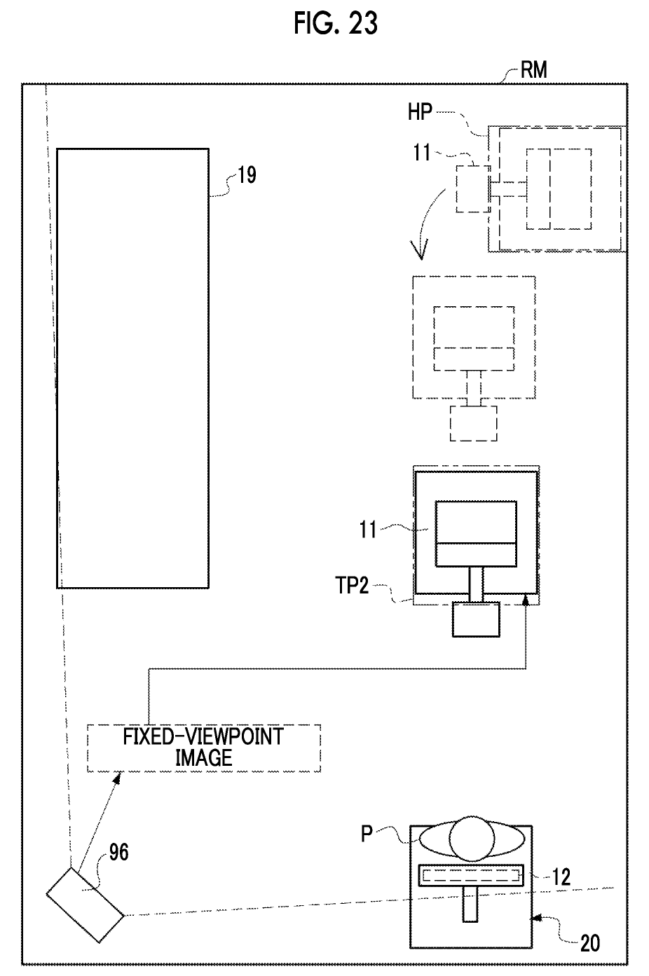

In addition, as shown in FIG. 23, the processor 40 may acquire a fixed-viewpoint image representing the surrounding environment of the radiation generation apparatus 11 captured by a fixed-point camera 96 provided at a fixed position, and execute the driving control based on the driving control image 70 and the fixed-viewpoint image. The fixed-point camera 96 is fixed at a fixed position in the imaging room RM, for example, and can include the entire region in which the radiation generation apparatus 11 moves in the angle of view. The fixed-viewpoint image is, for example, a video image, and can image a moving object that moves in the moving path of the radiation generation apparatus 11. In a case in which the feature point FP is tracked based on the driving control image 70, the moving object other than the radiation generation apparatus 11 is noise in the self-position estimation. Therefore, the processor 40 can perform the self-position estimation with high accuracy by specifying the moving object other than the radiation generation apparatus 11 from the fixed-viewpoint image and excluding the specified moving object as noise. As described above, such a noise may be removed in addition to the control of the frame rate according to the driving state.

In addition, the above-described embodiment is an example, and can be appropriately changed as follows.

The carriage unit 16 may be omitted, and the wheels 15 may be directly attached to a lower portion of the body part 14.

The camera 32 may be attached to the arm 27 instead of the radiation source 13. Similarly, the camera 33 may be provided in the body part 14 instead of the carriage unit 16. A plurality of the cameras 32 and 33 may be provided.

The camera 32 and the camera 33 may be integrated into one camera. In this case, the orientation of the camera is set to an orientation in which the driving control image 70 can be captured at the accommodation position, and an orientation in which the alignment control image 85 can be captured at the imaging preparation position.

The environment information sensor is not limited to the example of the camera 33. A light detection and ranging (LiDAR) sensor, a time-of-flight (TOF) sensor, or the like may be used. In addition, an inertial measurement unit (IMU) in which an acceleration sensor and a gyro sensor are combined, an ultrasonic sensor, a radar sensor, a magnetic sensor, or the like may be used.

The information acquisition frequency is not limited to the frame rate of the camera 33. For example, the information acquisition frequency corresponding to the type of the environment information sensor, such as the acquisition frequency of the distance image in a case of LiDAR, is included.

In addition, the radiographic image detection device is not limited to the example of the electronic cassette 12. A computed radiography (CR) cassette may be used. In addition, the radiographic image detection device may be a radiation detector fixed to an imaging table. In addition, the subject is not limited to the example of the patient P. The subject may be a diseased animal such as a dog or a cat.

In addition, the radiation generation apparatus 11 has been described as the radiography apparatus, but the radiography apparatus may be a radiographic image detection device including a driving mechanism.

The above description discloses the following Supplementary notes.

### Supplementary Note 1

A radiography apparatus which is used for radiography, the radiography apparatus comprising:

a radiation source or a radiographic image detection device;

a driving mechanism that is autonomously drivable and that includes an environment information sensor which acquires surrounding environment information; and

a processor configured to control the driving mechanism and that is capable of controlling an information acquisition frequency of the environment information sensor according to a driving state of the driving mechanism.

### Supplementary Note 2

The radiography apparatus according to Supplementary Note 1,

wherein the driving state includes at least one of a movement direction or a moving speed.

### Supplementary Note 3

The radiography apparatus according to Supplementary Note 2,

wherein the driving state includes straight movement in which the movement direction does not change and rotational movement in which the movement direction changes, and

the straight movement includes forward and backward movement, lateral movement, and diagonal movement.

### Supplementary Note 4

The radiography apparatus according to Supplementary Note 3,

wherein in a case of the rotational movement, the processor sets the information acquisition frequency to be higher than the information acquisition frequency in a case of the straight movement.

### Supplementary Note 5

The radiography apparatus according to Supplementary Note 4,

wherein in a case of the lateral movement or the diagonal movement, the processor sets the information acquisition frequency to be higher than the information acquisition frequency in a case of the forward and backward movement.

### Supplementary Note 6

The radiography apparatus according to any one of Supplementary Notes 3 to 5,

wherein in a case where the rotational movement and the straight movement are performed in parallel, the processor sets the information acquisition frequency to be higher than the information acquisition frequency in a case where only the straight movement is performed.

### Supplementary Note 7

The radiography apparatus according to any one of Supplementary Notes 3 to 6,

wherein the processor controls the driving mechanism such that a final position adjustment to a target position is the straight movement.

### Supplementary Note 8

The radiography apparatus according to Supplementary Note 2,

wherein the processor sets the information acquisition frequency to be higher as the moving speed is faster.

### Supplementary Note 9

The radiography apparatus according to any one of Supplementary Notes 1 to 8,

wherein the processor is configured to:

acquire a fixed-viewpoint image representing a surrounding environment captured by a fixed-point camera provided at a fixed position; and

execute driving control based on environment information acquired from the environment information sensor and the fixed-viewpoint image.

### Supplementary Note 10

The radiography apparatus according to any one of Supplementary Notes 1 to 9,

wherein the environment information sensor is a camera that images a surrounding environment, and

the information acquisition frequency is a frame rate.

### Supplementary Note 11

The radiography apparatus according to any one of Supplementary Notes 1 to 10,

wherein the radiography apparatus is a radiation generation apparatus including the radiation source.

### Supplementary Note 12

An operation method of a radiography apparatus which is used for radiography, and which includes a radiation source or a radiographic image detection device, a driving mechanism that is autonomously drivable and that includes an environment information sensor which acquires surrounding environment information, and a processor configured to control the driving mechanism, the operation method comprising:

controlling, by the processor, an information acquisition frequency of the environment information sensor according to a driving state of the driving mechanism.

### Supplementary Note 13

An operation program of a radiography apparatus which is used for radiography, and which includes a radiation source or a radiographic image detection device, a driving mechanism that is autonomously drivable and that includes an environment information sensor which acquires surrounding environment information, and a processor configured to control the driving mechanism, the operation program causing the processor to execute a process comprising:

controlling an information acquisition frequency of the environment information sensor according to a driving state of the driving mechanism.

In the above-described embodiment, the processing executed by the processor 40 is executed by any computer. In addition, any computer may execute these types of processing by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate.

Any computer may be a general-purpose computer, a computer for specific use, a workstation, or another system capable of executing each processing. The processor may be configured using one or more pieces of hardware, and the type of hardware is not limited. For example, the processor may be configured by a programmable logic device such as a central processing unit (CPU), a micro processing unit (MPU), or a field programmable gate array (FPGA), a dedicated circuit for executing specific processing, such as an application specific integrated circuit (ASIC), or hardware such as a graphic processing unit (GPU) or a neural processing unit (NPU). Furthermore, the types of hardware may be a combination of different types of hardware. In a case in which the plurality of types of hardware are configured to execute one or a plurality of types of processing of a certain processor, the plurality of types of hardware may exist in devices physically separated from each other or may exist in the same device. Furthermore, in any of the embodiments, the order of each processing performed by the processor is not limited to the above-described order, and may be changed as appropriate. The hardware is configured by an electric circuit (circuitry) in which circuit elements such as semiconductor elements are combined.

The program may be software such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium or other storage). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segments may be connected to other code segments or hardware circuits by transmitting and receiving information, data, an argument, a parameter, or content of a memory.

The technology of the present disclosure can also be combined with various embodiments and/or various modification examples described above, as appropriate. In addition, the present disclosure is not limited to the above-described embodiments, and various configurations can be adopted without departing from the gist of the present disclosure. Further, the technology of the present disclosure includes a storage medium that stores the program in a non-transitory manner, in addition to the program. The storage medium is, for example, a non-transitory computer-readable storage medium such as a universal serial bus (USB) memory, a flexible disk, or a compact disc read only memory (CD-ROM). The program may be provided online through a network such as the Internet. The disclosed technology also applies to a program product in addition to the program. The program product includes products of every aspect for providing the program. Like the program, the program product may be provided by being stored in a non-transitory computer-readable storage medium or may be provided online.

The above descriptions and illustrations are detailed descriptions of portions related to the technology of the present disclosure and are merely examples of the technology of the present disclosure. For example, the above description of the configuration, the function, the operation, and the effect are the description of examples of the configuration, the function, the operation, and the effect of the parts according to the technology of the present disclosure. Accordingly, it goes without saying that unnecessary parts may be deleted, new elements may be added, or replacements may be made with respect to the above-described contents and the above-shown contents within a range that does not deviate from the gist of the technology of the present disclosure. In addition, in the above descriptions and illustrations, the description of, for example, common technical knowledge that does not need to be particularly described to enable the implementation of the technology of the present disclosure is omitted in order to avoid confusion and facilitate the understanding of portions related to the technology of the present disclosure.

In the specification, "A and/or B" is synonymous with "at least one of A or B". That is, "A and/or B" means that it may be only A, only B, or a combination of A and B. Further, in the specification, the same concept as "A and/or B" is applied to a case in which the connection of three or more matters is expressed by "and/or".

All of the documents, the patent applications, and the technical standards described in the specification are incorporated by reference herein to the same extent as each document, each patent application, and each technical standard are specifically and individually stated to be incorporated by reference.

What is claimed is:

1. A radiography apparatus which is used for radiography, the radiography apparatus comprising:
a radiation source or a radiographic image detection device;
a driving mechanism that is autonomously drivable and that includes an environment information sensor which acquires surrounding environment information; and
a processor configured to control the driving mechanism and that is capable of controlling an information acquisition frequency of the environment information sensor according to a driving state of the driving mechanism.

2. The radiography apparatus according to claim 1,
wherein the driving state includes at least one of a movement direction or a moving speed.

3. The radiography apparatus according to claim 2,
wherein the driving state includes straight movement in which the movement direction does not change and rotational movement in which the movement direction changes, and
the straight movement includes forward and backward movement, lateral movement, and diagonal movement.

4. The radiography apparatus according to claim 3,
wherein in a case of the rotational movement, the processor sets the information acquisition frequency to be higher than the information acquisition frequency in a case of the straight movement.

5. The radiography apparatus according to claim 4,
wherein in a case of the lateral movement or the diagonal movement, the processor sets the information acquisition frequency to be higher than the information acquisition frequency in a case of the forward and backward movement.

6. The radiography apparatus according to claim 3,
wherein in a case where the rotational movement and the straight movement are performed in parallel, the processor sets the information acquisition frequency to be higher than the information acquisition frequency in a case where only the straight movement is performed.

7. The radiography apparatus according to claim 3,
wherein the processor controls the driving mechanism such that a final position adjustment to a target position is the straight movement.

8. The radiography apparatus according to claim 2,
wherein the processor sets the information acquisition frequency to be higher as the moving speed is faster.

9. The radiography apparatus according to claim 1,
wherein the processor is configured to:
acquire a fixed-viewpoint image representing a surrounding environment captured by a fixed-point camera provided at a fixed position; and
execute driving control based on environment information acquired from the environment information sensor and the fixed-viewpoint image.

10. The radiography apparatus according to claim 1,
wherein the environment information sensor is a camera that images a surrounding environment, and
the information acquisition frequency is a frame rate.

11. The radiography apparatus according to claim 1,
wherein the radiography apparatus is a radiation generation apparatus including the radiation source.

12. An operation method of a radiography apparatus which is used for radiography, and which includes a radiation source or a radiographic image detection device, a driving mechanism that is autonomously drivable and that includes an environment information sensor which acquires surrounding environment information, and a processor configured to control the driving mechanism, the operation method comprising:
controlling, by the processor, an information acquisition frequency of the environment information sensor according to a driving state of the driving mechanism.

13. A non-transitory computer-readable storage medium storing an operation program of a radiography apparatus which is used for radiography, and which includes a radiation source or a radiographic image detection device, a driving mechanism that is autonomously drivable and that includes an environment information sensor which acquires surrounding environment information, and a processor configured to control the driving mechanism, the operation program causing the processor to execute a process comprising:
controlling an information acquisition frequency of the environment information sensor according to a driving state of the driving mechanism.
